(19) United States
(12) Patent Application Publication
(10) Pub. No.: US 2018/0021503 A1
(43) Pub. Date: Jan. 25, 2018

(54) VAGINAL DOUCHE WITH A COMPOSITION TO TIGHTEN THE VAGINA

(71) Applicant: Erica Stewart, Menifee, CA (US)

(72) Inventor: Erica Stewart, Menifee, CA (US)

(21) Appl. No.: 15/213,460

(22) Filed: Jul. 19, 2016

(51) Int. Cl. A61M 3/02; A61K 36/28; A61K 9/00; A61K 35/747; A61K 33/30; A61K 31/185; A61K 36/73; A61K 31/047

(52) U.S. Cl. CPC A61M 3/0279; A61K 36/73; A61K 36/28; A61K 31/047; A61K 35/747; A61K 33/30; A61K 31/185; A61K 9/0034; A61K 2035/11

(57) ABSTRACT

A vaginal douche bottle with a composition of ingredients to tighten and prevention of growth of bad microorganisms in reproductive organs of human is provided. The vaginal douche bottle includes a removable cap, a middle portion, a bottom portion, and threads. The removable cap is adapted to remove from the vaginal douche bottle when a pipe adapter is required to be coupled with the vaginal douche bottle. The middle portion allows a user to hold the vaginal douche bottle. Threads are grooved on a top portion of the vaginal douche bottle to couple with a lower portion of the pipe adapter. The composition of ingredients filled in the vaginal douche bottle. When the middle portion of the vaginal douche bottle is pressed, the composition of ingredients comes out from the vaginal douche bottle through the adapter pipe and cleans the reproductive organs of the user.

### BACKGROUND

### Technical Field

The embodiments herein generally relate to a feminine hygiene product, and, more particularly, a vaginal douche with a composition to tighten vagina.

### Description of the Related Art

Child birth leads to the stretching of vaginal tissues. During the child birth, the baby travels through the cervix and out through the vagina, leading to the weakening of vaginal tissue. Sometimes the skin between the vagina and anus (i.e., the perineum) tear up or intentionally cut by doctor for allowing the normal birth of baby without any difficulty. Regular sexual intercourse is another cause of loose vaginal walls.

Females suffer problems like vaginal diseases and bad odor due to the growth of bad microorganisms. The vaginal diseases spread due to growth of bad bacteria, fungus, yeast, which grow by replacing the good micro flora near the vaginal area of females. The vaginal diseases lead to vaginal discharge, inflammation, and irritation, as well as vulvar or vaginal itching in females. Sometimes the vaginal diseases are so fatal that leads to death of the person.

There are surgical methods present to tighten the vagina. But the procedure is costly, time consuming and require proper rest for longer period of time. For preventing the growth of bad bacteria, medical compositions are available in the market. But the medicines are not much effective and the cost of these medicines is very high and has side effects like nausea, fever, and cough or other similar fatal symptoms.

Accordingly, there remains a need of a simple solution to tighten the vagina and to prevent the vaginal problems without any surgical methods.

### SUMMARY

In view of the foregoing an embodiment herein provides a vaginal douche bottle with a composition of ingredients to tighten vagina and prevents growth of bad microorganisms in reproductive organs of human. The vaginal douche bottle includes a removable cap, a middle portion, a bottom portion, and one or more threads. The removable cap is adapted to remove from the vaginal douche bottle when a pipe adapter is required to be coupled with the vaginal douche bottle. The middle portion allows a user to hold the vaginal douche bottle. The bottom portion provides a support to the vaginal douche bottle. The one or more threads are grooved on a top portion of the vaginal douche bottle to couple with the lower portion of the pipe adapter. The composition of ingredients filled in the vaginal douche bottle.

When the middle portion of the vaginal douche bottle is pressed, the composition of ingredients comes out from the vaginal douche bottle through the adapter pipe and cleans & tightens the reproductive organs of the user. In one embodiment, The composition includes a mixture of (i) apple cider vinegar in an amount of 237 ml; purified water in an amount of 118 ml; echinacea in an amount of 1 teaspoon; (iv) clendula extract in an amount of 1 teaspoon; (v) vegetable glycerin in an amount of 1/₂ teaspoon; (vi) Lactobacillus acidophilus in an amount of 15 ml; (vii) zinc sulfate in an amount of 1 teaspoon; and (viii) benzenesulfonic acid in an amount of 1 teaspoon. The property of the composition is to tighten the vagina by rejuvenating the damaged vaginal cells and to prevent the bad odor by preventing the growth of bad microorganisms.

These and other aspects of the embodiments herein will be better appreciated and understood when considered in conjunction with the following description and the accompanying drawings. It should be understood, however, that the following descriptions, while indicating preferred embodiments and numerous specific details thereof are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a diagrammatic representation of the vaginal douche bottle with a composition of ingredients to maintain the pH of vaginal area and prevent the growth of bad microorganisms according to an embodiment herein;

FIG. 2 illustrates a diagrammatic representation of the vaginal douche bottle when the removable cap is removed according to an embodiment herein;

FIG. 3 illustrates a diagrammatic representation of the pipe adapter of FIG. 1 according to an embodiment herein;

FIG. 4 illustrates a tabulated view of the composition of the ingredients of FIG. 1 according to an embodiment herein;

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

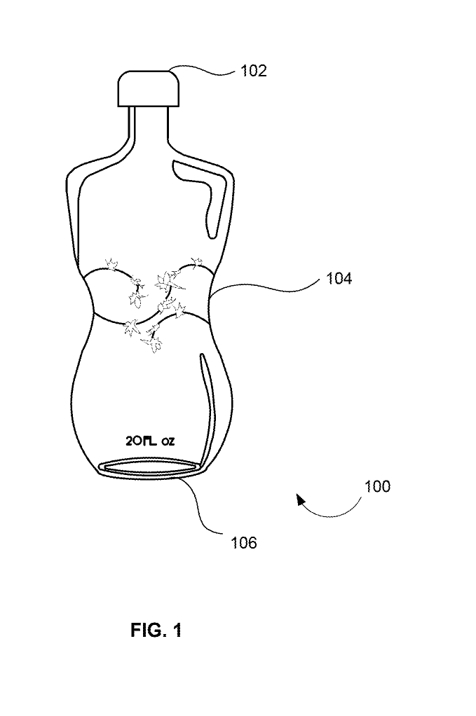

As mentioned, there remains a need for cheap, easy to use, preventive and non-toxic feminine cleansing solution in a nonsurgical method to maintain vaginal health. The embodiments herein achieve this by providing a vaginal douche with a liquid composition. Referring now to the drawings, and more particularly to FIGS. 1 through 4, where similar reference characters denote corresponding features consistently throughout the figures, there are shown preferred embodiments.

FIG. 1 illustrates a diagrammatic representation of the vaginal douche bottle 100 with a composition of ingredients to maintain the pH of vaginal area and prevent the growth of bad microorganisms according to an embodiment herein. The vaginal douche bottle 100 with the composition of ingredients is for balancing the pH of vaginal area within a range from 3.5 to 4.5. The vaginal douche bottle 100 includes a removable cap 102, a middle portion 104, and a bottom portion 106. The removable cap 102 is provided to keep the vaginal douche bottle 100 closed, when not in use. The composition of ingredients is filled in the vaginal douche bottle 100 in the form of liquid. When a user press the middle area 104 of the vaginal douche bottle 100, the composition of ingredients is come out from the vaginal douche bottle 100 through the adapter pipe (not shown in FIG. 1) and cleans the reproductive organs of the user. The composition includes, but not limited, apple cider vinegar, vegetable, glycerin, Echinacea, purified water, and/or mixture thereof.

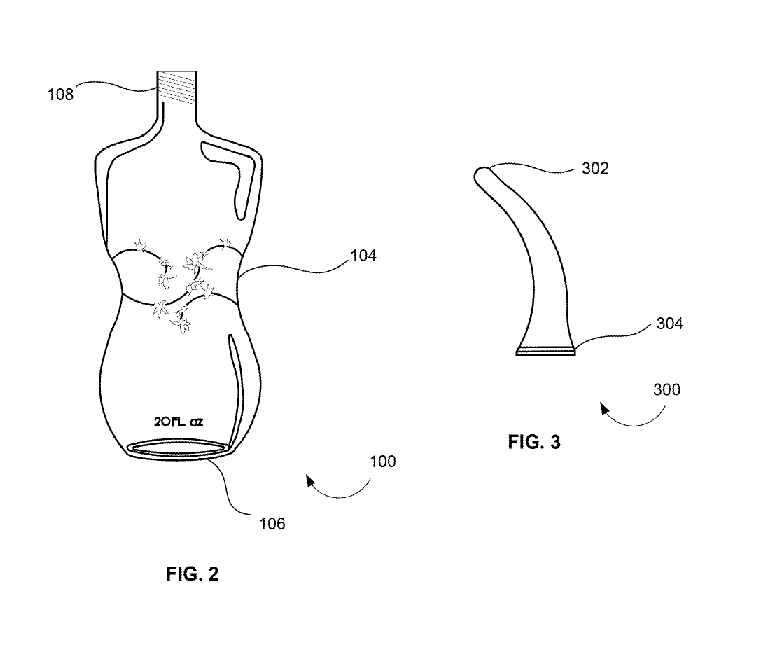

FIG. 2 illustrates a diagrammatic representation of the vaginal douche bottle 100 when the removable cap 102 is removed from the vaginal douche bottle 100 according to an embodiment herein. The vaginal douche bottle 100 further includes one or more threads 108 that couples with the pipe adapter (not shown in FIG. 2). The pipe adapter is adapted to couple with the one or more threads 108 of the vaginal douche bottle 100. When the user press the middle area 104 of the vaginal douche bottle 100, the composition of ingredients comes out from the vaginal douche bottle 100 through the adapter pipe (not shown in FIG. 2) and cleans the reproductive organs of the user. In one embodiment, the vaginal douche bottle 100 is manufactured from the material of plastic, metal, rubber and/or mixture thereof. The property of the composition is to tighten the vagina by rejuvenating the damaged vaginal cells and to prevent the bad odor by preventing the growth of bad microorganisms.

FIG. 3 illustrates a diagrammatic representation of the pipe adapter 300 of FIG. 1 according to an embodiment herein. The pipe adapter 300 includes having a tip portion 302, and a lower portion 304. The tip portion 302 is adapted to get touch with the reproductive organs of the user. The lower portion 304 is adapted to couple with the one or more threads 108 of the vaginal douche bottle 100. The lower portion 304 further includes one or more threads, which helps in coupling the pipe adapter 300 with the vaginal douche bottle 100. In one embodiment, the pipe adapter 300 is made from a material selected from a group of polyethylene, Teflon, stainless steel, other metals and/or mixture thereof.

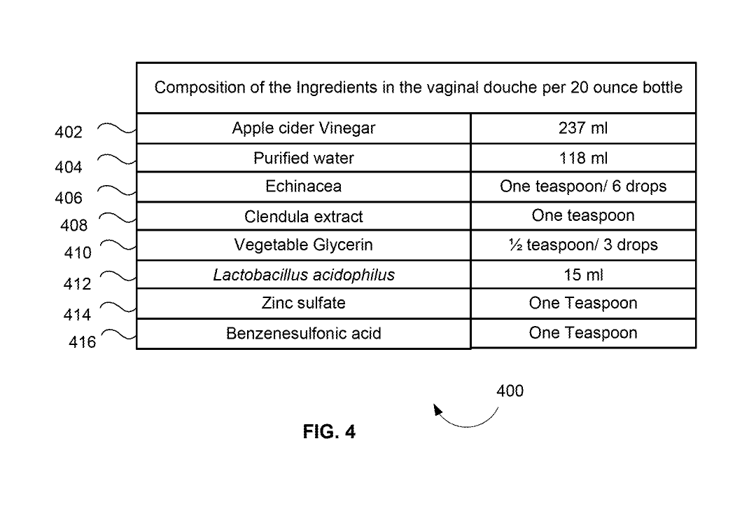

FIG. 4 illustrates a tabulated view 400 of the composition of the ingredients of FIG. 1 according to an embodiment herein. The tabulated view 400 depicts the composition of the ingredients in the vaginal douche per 20 ounce bottle. The composition includes a mixture of apple cider vinegar 402, purified water 404, echinacea 406, clendula extract 408, vegetable glycerin 410, lactobacillus acidophilus 412, zinc sulfate 414, benzenesulfonic acid 416, and/or mixture thereof. The apple cider vinegar is used in the amount “237 ml” to tighten and refresh the vagina. The purified water is used in amount “118 ml” function is to clean the vagina. The echinacea is used in amount “1 teaspoon/6 drops” to kill the bad microorganisms, without harming good microorganisms. The clendula extract used in the amount “1 teaspoon”, vegetable glycerin is used in the amount ½ teaspoon/3 drops to moisture the vagina. The Lactobacillus acidophilus is used in the amount 15 ml. The zinc sulfate is used in the amount “1 teaspoon” and benzenesulfonic acid is used in the amount “1 teaspoon”.

The vaginal douche bottle 100 with the composition of ingredients helps in tightening of vagina and prevents the growth of bad micro organisms in reproductive organs of human. The composition helps in preventing the bad odor or foul smell due to bad bacteria. The apple cider vinegar tightens the vagina walls without rejuvenation surgery. The Echinacea helps in killing all the bad microorganisms and leaving good bacteria in place in the vagina. The purified water helps to clean the vagina. The vegetable glycerin adds moisture to the vagina.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A vaginal douche bottle with a composition of ingredients to tighten and prevention of growth of bad microorganisms in reproductive organs of human, wherein said vaginal douche bottle comprises:
a removable cap that is adapted to remove from vaginal douche bottle when a pipe adapter is required to be coupled with said vaginal douche bottle;
a middle portion that allows a user to hold said vaginal douche bottle;
a bottom portion that provides a support to whole said vaginal douche bottle; and
a plurality of threads that are grooved on a top portion of said vaginal douche bottle to couple with a lower portion of said pipe adapter;
wherein said composition of ingredients filled in said vaginal douche bottle, wherein when said middle portion of said vaginal douche bottle is pressed, said composition of ingredients comes out from said vaginal douche bottle through said adapter pipe and cleans the reproductive organs of said user.

2. The vaginal douche bottle of claim 1, wherein said composition of ingredients comprises a mixture of apple cider vinegar, purified water, Echinacea, clendula extract, vegetable glycerin, Lactobacillus acidophilus, zinc sulfate, and benzenesulfonic acid.

3. The vaginal douche bottle of claim 2, wherein said apple cider vinegar is in an amount of 237 ml in said mixture.

4. The vaginal douche bottle of claim 2, wherein said purified water is in an amount of 118 ml in said mixture.

5. The vaginal douche bottle of claim 2, wherein said echinacea is in an amount of 1 teaspoon in said mixture.

6. The vaginal douche bottle of claim 2, wherein said clendula extract is in an amount of 1 teaspoon in said mixture.

7. The vaginal douche bottle of claim 2, wherein said vegetable glycerin is in an amount of ½ teaspoon in said mixture.

8. The vaginal douche bottle of claim 2, wherein said Lactobacillus acidophilus is in an amount of 15 ml in said mixture.

9. The vaginal douche bottle of claim 2, wherein said zinc sulfate is in an amount of 1 teaspoon in said mixture.

10. The vaginal douche bottle of claim 2, wherein said benzenesulfonic acid is in an amount of 1 teaspoon in said mixture.

11. A composition of ingredients to tighten and prevention of growth of bad microorganisms in reproductive organs of human, wherein said composition comprises a mixture of
apple cider vinegar in an amount of 237 ml;
purified water in an amount of 118 ml;
echinacea in an amount of 1 teaspoon;
clendula extract in an amount of 1 teaspoon;
vegetable glycerin in an amount of ½ teaspoon;
Lactobacillus acidophilus in an amount of 15 ml;
zinc sulfate in an amount of 1 teaspoon; and
and benzenesulfonic acid in an amount of 1 teaspoon.
